(19) United States
(12) Patent Application Publication
(10) Pub. No.: US 2017/0332917 A1
(43) Pub. Date: Nov. 23, 2017

(54) NON-INTRUSIVE PORTABLE SLEEP APNEA ASSESSMENT SYSTEM

(71) Applicant: THE REGENTS OF THE UNIVERSITY OF CALIFORNIA, Oakland, CA (US)

(72) Inventors: Majid Sarrafzadeh, Anaheim Hills, CA (US); Lauren E. Samy, Los Angeles, CA (US)

(73) Assignee: THE REGENTS OF THE UNIVERSITY OF CALIFORNIA, Oakland, CA (US)

(21) Appl. No.: 15/600,564

(22) Filed: May 19, 2017

Related U.S. Application Data
(60) Provisional application No. 62/338,890, filed on May 19, 2016.

(51) Int. Cl. A61B 5/0205; A61B 5/024; A61B 5/00; A61B 5/08; G06F 19/00; G06F 3/01

(52) U.S. Cl. CPC A61B 5/0205; G06F 19/3418; G06F 3/011; A61B 5/4818; A61B 5/0826; A61B 5/0816; A61B 5/0004; A61B 5/0803; A61B 5/024; A61B 5/08; A61B 5/0002

(57) ABSTRACT

A method of sleep apnea diagnosis includes providing interrogatories at a user interface; receiving responses to the interrogatories at the user interface; receiving blood pressure measurement information from a blood pressure monitoring device; receiving heart rate measurement information from a heart rate monitoring device; and determining, from the responses to the interrogatories, the blood pressure measurement information and the heart rate measurement information, a classification of a subject, the classification being either having sleep apnea disorder or not having sleep apnea disorder.

### CROSS-REFERENCE TO RELATED APPLICATIONS

This application claims the benefit of and priority to U.S. Provisional Patent Application 62/338,890, filed May 19, 2016, which is incorporated herein by reference in its entirety.

### BACKGROUND

Quantity and quality of sleep have a large impact on health and well-being. Repair and regeneration tasks are carried out during sleep to maintain physical, mental and emotional health. Obstructive sleep apnea (OSA) is a sleep disorder that is characterized by repeated pauses in breathing during sleep. These pauses, also referred to as apneas, deplete the brain and the rest of the body of oxygen and disrupt the normal sleep cycle. The apneas can result in memory impairment, cognitive changes, and excessive daytime sleepiness and fatigue. A technique for diagnosing OSA is polysomnography (PSG). PSG is an overnight sleep test which monitors biophysical changes (e.g., electroencephalogram (EEG), electrocardiogram (ECG), etc.) of a subject during sleep. However, PSG is expensive and intrusive, and can produce inaccurate data due to intrinsic imperfections of the experimental setup.

### SUMMARY

In the present disclosure, at least some embodiments relate to a sleep apnea screening system and technology. Results of sleep apnea studies using the systems and technology of the present disclosure are also provided. The sleep apnea screening system and technology incorporate advanced feature selection and machine learning to build effective prediction models that assist in identifying sleep apnea sufferers.

In some embodiments, a method of sleep apnea diagnosis comprises providing interrogatories at a user interface; receiving responses to the interrogatories at the user interface; receiving blood pressure measurement information from a blood pressure monitoring device; receiving heart rate measurement information from a heart rate monitoring device; and determining, from the responses to the interrogatories, the blood pressure measurement information and the heart rate measurement information, a classification of a subject, the classification being either having sleep apnea disorder or not having sleep apnea disorder.

In some embodiments, a system for sleep apnea diagnosis comprises a user interface, a blood pressure monitoring device, a heart rate monitoring device, a data storage component and a classifier. The user interface is configured to provide interrogatories and receive responses to the interrogatories. The blood pressure monitoring device is configured to collect blood pressure measurement information. The heart rate monitoring device is configured to collect heart rate measurement information. The data storage component is configured to store the responses to the interrogatories, the blood pressure measurement information, and the heart rate measurement information. The classifier is configured to determine a classification of a subject, the classification being either having sleep apnea disorder or not having sleep apnea disorder, based on the responses to the interrogatories, the blood pressure measurement information and the heart rate measurement information.

In some embodiments, a sleep apnea diagnostic system comprises a computing device configured to execute instructions from a memory to: provide interrogatories at a user interface; receive responses to the interrogatories at the user interface; receive blood pressure measurement information from a blood pressure monitoring device; receive heart rate measurement information from a heart rate monitoring device; and determine, from the responses to the interrogatories, the blood pressure measurement information and the heart rate measurement information, a classification of a subject, the classification being either having sleep apnea disorder or not having sleep apnea disorder.

### BRIEF DESCRIPTION OF THE DRAWINGS

Aspects of some embodiments of the present disclosure are best understood from the following detailed description when read with the accompanying figures. It is noted that various structures may not be drawn to scale, and dimensions of the various structures may be arbitrarily increased or reduced for clarity of discussion.

FIG. 1 illustrates an example of an OSA screening system architecture according to embodiments of the present disclosure.

FIG. 2 illustrates an example of an OSA screen according to embodiments of the present disclosure.

FIG. 3 illustrates an example of variance for individual predictions and overall average variance of a k-nearest-neighbor classifier of an OSA screen according to embodiments of the present disclosure.

FIG. 4 illustrates accuracy, sensitivity, and specificity of an OSA screen classification according to embodiments of the present disclosure.

FIG. 5 illustrates receiver operating characteristic curves for gender-agnostic and gender-aware OSA screen classifications according to embodiments of the present disclosure.

FIG. 6 illustrates an example of a computing device according to embodiments of the present disclosure.

### DETAILED DESCRIPTION

Common reference numerals are used throughout the drawings and the detailed description to indicate the same or similar components. Embodiments of the present disclosure will be readily understood from the following detailed description taken in conjunction with the accompanying drawings.

The following disclosure provides many different embodiments, or examples, for implementing different features of the provided subject matter. Specific examples of components and arrangements are described below to explain certain aspects of the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

Sleep apnea is a sleep disorder that affects tens of millions of people in the United States and worldwide. The most common type of sleep apnea is called obtrusive sleep apnea (OSA). In OSA, the tongue and throat muscles relax during sleep along with other body muscles. For OSA sufferers, the relaxed tongue and throat muscles press against the soft tissue in the upper airway of the throat, causing a partial or complete blockage of the airway, which can cause an OSA sufferer to stop breathing or to experience abnormally low-volume breathing. Each pause in breathing can last, e.g., from a few seconds to a few minutes, and may occur, e.g., several times per hour. After unsuccessfully breathing, the brain of an OSA sufferer starts suffering from lack of oxygen and wakes the OSA sufferer up temporarily to gasp for air and resume a normal breathing pattern. When the OSA sufferer falls back asleep, the muscles start to relax again and the whole cycle repeats. Some OSA sufferers may repeat this cycle hundreds of times in one night. The OSA severely disrupts the normal sleep cycle and the corresponding repair and regeneration associated with sleep.

Because of the fragmented sleep pattern, OSA sufferers experience excessive daytime sleepiness and fatigue. Also, the continuous reduction in blood oxygenation stresses the OSA sufferer's physical system, leading to comorbidities such as hypertension, congestive heart failure, and depression. An individual with OSA is rarely aware of having difficulty breathing and often cannot remember the brief breathing-related arousals during the night. As a result, many people remain undiagnosed and untreated. It is estimated that less than twenty-five percent (%) of OSA sufferers are diagnosed.

A diagnostic tool presently available for determining the presence of sleep apnea is polysomnography (PSG). A polysomnogram or “sleep study” is a costly overnight sleep test, during which participants spend the night at a sleep laboratory and biophysiological signals are monitored during sleep. Many different sensors can be attached to the participant's body to measure, e.g., brain waves (through, e.g., electroencephalogram (EEG)), eye movements (through, e.g., electrooculography (EOG)), muscle activity (through, e.g., electromyography (EMO)), heart rhythm (through, e.g., electrocardiogram (ECG)), oral and nasal airflow, blood oximetry, and audio during sleep. The nature of polysomnography, however, can interfere with the results and accuracy of the measured signals. For example, the unfamiliar environment of the sleep laboratory and the equipment and sensors attached to the participant's body can disturb the participant's quality of sleep. Further, the prohibitive cost of the sleep test discourages many people from being diagnosed.

According to at least some embodiments of the present disclosure describe an affordable, fast and non-intrusive sleep-apnea detection system and technology that can accurately screen for sleep apnea (e.g., OSA). In some embodiments, the disclosed technology does not involve specialized equipment (e.g., equipment used in a PSG study). Thus, a person can perform the screen in his/her own environment or at healthcare professional's office rather than at a sleep laboratory. In some embodiments, the operation of the system is straightforward and the person can set up the screen process without help from others. In some embodiments, the sleep apnea screening of the present disclosure can be performed while the person is awake. In some embodiments, the sleep apnea screening may have a duration of, e.g., approximately 15-20 minutes, in contrast to the PSG which is a full-night test. Not only does the daytime test avoid interruption of the person's sleep, but the short test duration also significantly reduces the amount of data collected and used in a sleep apnea analysis as compared to an overnight test.

Sleep apnea can be accompanied by brain injury as a result of oxygen depletion to the brain. Because functions of the brain include regulating blood pressure and blood circulation, sleep apnea can be associated with a dysfunction in cardiovascular regulation, and particularly with respect to heart rate response. Persons suffering sleep apnea can have heart rate responses that are delayed and less pronounced compared to healthy persons. In some embodiments, the sleep apnea screening system and technology of the present disclosure predict the presence or lack of sleep apnea by triggering and observing cardiovascular responses.

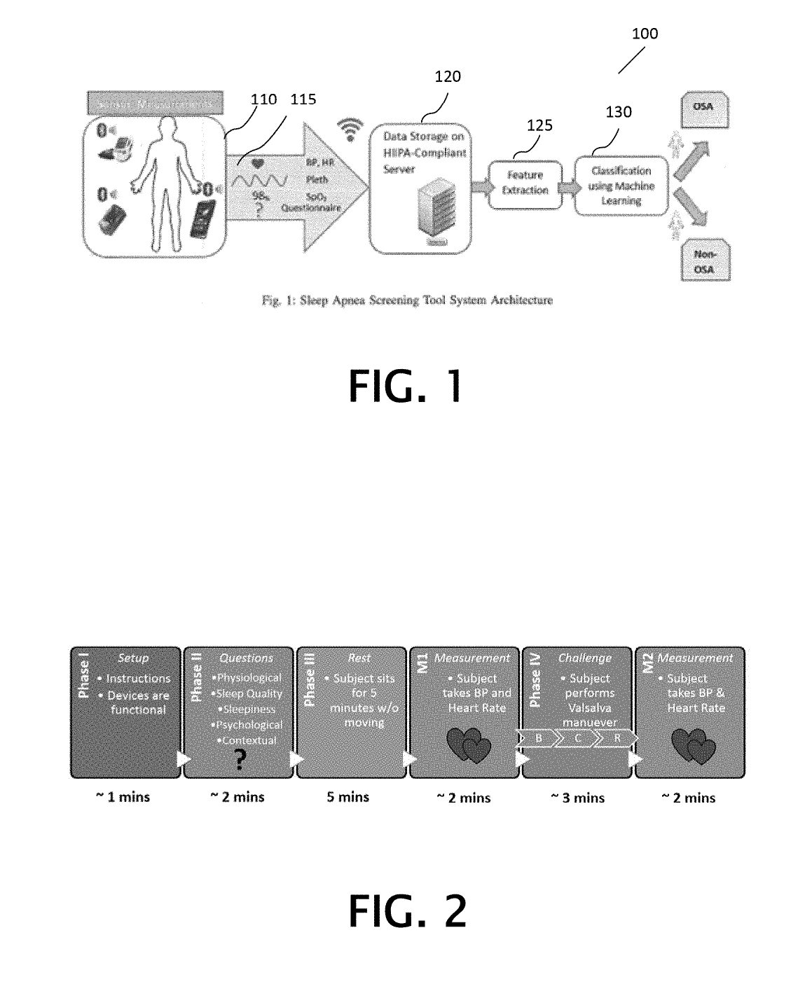

FIG. 1 illustrates an example of an OSA screening architecture 100 according to embodiments of the present disclosure. The OSA screening architecture 100 (also referred to as “disclosed system” or simply “system”) includes an OSA diagnostic system 110 that receives information from a subject and provides the information externally. The information is provided by way of a network 115 to a central storage device 120 (e.g., a Health Insurance Portability and Accountability Act (HIPAA)-compliant computing device). The information is analyzed to extract features, and classified as OSA or non-OSA using a machine learning technique.

The OSA diagnostic system 110 includes an interactive triggering technique that guides the subject through a series of challenges that have been medically identified as triggering particular cardiovascular responses.

FIG. 2 illustrates an example of an OSA screen performed by the OSA diagnostic system 110. In some embodiments, the OSA screen may last, e.g., approximately 15-20 minute. The OSA screen in the embodiment illustrated in FIG. 2 includes three phases (Phase I, Phase II and Phase III) and two measurement periods (M1 and M2).

In a first phase (Phase I), the OSA screen is set up. The subject is interrogated to determine whether the subject can perform the OSA screen in an uninterrupted manner, whether a blood pressure (BP) cuff is on and whether a Valsalva maneuver box (described below) to be used in the OSA screen has been pre-tested.

In a second phase (Phase II), the subject is further interrogated with a sequence of questions, examples of which are listed in Table I. The answers to the interrogatories are provided to the storage device 120.

TABLE I

| Measure | QID | Question |
|---|---|---|
| Sleep | q_l | What time did you go to bed last night? |
| Quality | q_2 | Approximately how long did it take for you to |
| | | get to sleep? |
| | q_3 | Approximately how many hours did you sleep? |
| | q_4 | What time did you get out of bed this morning? |
| Sleepiness | q_5 | Rate your sleepiness today (0 = none, 10 = |
| | | extremely high). |
| Perceived | q_7 | In the last day, how often have you felt that you |
| Stress | | were unable to control the important things in |
| | | your life? |
| | q_8 | In the last day, how often have you felt confident |
| | | about your ability to handle your personal |
| | | problems? |
| | q-9 | In the last day, how often have you felt that things |
| | | were going your way? |
| | q_10 | In the last day, how often have you felt difficul- |
| | | ties were piling up so high that you could not |
| | | overcome them? |
| Depression | q_ll | In the last day, how often have you experienced |
| | | little interest or little pleasure in doing things? |
| | q_l2 | In the last day, how often have you felt down, |
| | | depressed or hopeless? |
| Anxiety | q_l3 | In the last day, how often have you felt nervous, |
| | | anxious or on edge? |
| | q_l4 | In the last day, how often have you not been able |
| | | to stop or control worrying? |
| Context | q_6 | Did you experience anything out of your normal |
| | | routine in the past 24 hours? |
| | q_l5 | Rate your level of physical activity over the last |
| | | 24-hour period. |

In a third phase (Phase III), following the interrogatories, the subject is instructed to rest for a period, such as for five minutes. The end of the rest period is noted with an indicator (e.g., a beeping sound or a light).

In a first measurement period M1 after the rest period of Phase III, the subject is instructed to take a baseline BP measurement (bp_baseline) and a resting heart rate baseline (HR_baseline). For example, the instructions to take the baseline BP measurement and the resting heart rate baseline may include instructions for positioning the BP cuff, inflating the BP cuff, placing a sensor for BP measurement, taking of a heart rate measurement, and other instructions. The baseline BP measurement and the resting heart rate baseline are provided to the storage device 120.

In a fourth phase (Phase IV), the subject is provided with instructions for completing, e.g., three challenges (Challenge 1, Challenge 2, Challenge 3) according to the triggering technique. Each challenge triggers a cardiovascular response of interest for OSA detection.

In some embodiments, Challenge 1 may be a Stroop test where the subject is asked, under time pressure, to determine the name of a color that is displayed in a color not denoted by the name (e.g., respond “red” when seeing the word “blue” printed in red). Because naming a color displayed using a mismatched pair of descriptor and color requires more attention and is more error-prone than when the color and the descriptor of the color match, the Stroop test may serve as a psychological and cognitive stressor that can induce heightened levels of physiological responses.

In some embodiments, Challenge 2 may be a Valsalva maneuver where the subject blows against a closed airway connected to a pressure measuring device. For example, the subject is instructed to exhale for about 18 seconds at a pressure of about 40 millimeters mercury (mm Hg). A Valsalva maneuver box as used in investigations as described in the present disclosure may include a blue light that illuminates at about 40 mm Hg. The Valsalva maneuver may result in changes in blood pressure and heart rate and may be used to evaluate the condition of the heart. Because OSA may be accompanied by brain injury as a result of oxygen depletion to the brain, and one of the functions of the brain is to regulate blood pressure and blood circulation, OSA may be associated with a dysfunction in the cardiovascular regulation. The OSA diagnostic system 110 can compare cardiovascular responses caused by the Valsalva maneuver as signaled by an injured brain to cardiovascular responses signaled by a healthy brain. The comparison may provide clues to the existence of OSA.

In some embodiments, Challenge 3 may be a breath holding exercise where the subject is instructed to hold his/her breath for about 30 seconds. This challenge acts as a simulation of an apneic attack and triggers changes in heart rate and blood pressure that occur after such an apneic attack.

It should be noted that in some embodiments, instead of three challenges, less than three challenges (i.e., one or two challenges) are included in the OSA screen; in some embodiments, more than three challenges are included in the OSA screen. Each of the challenges (e.g., Challenge 1, Challenge 2, and Challenge 3) may be preceded by a baseline period of rest (e.g., approximately 60 seconds) and may be followed by a recovery period of rest (e.g., approximately 90 seconds), to reach a normal or resting blood pressure and to prepare for the next challenge or measurement.

After Phase IV (e.g., after the recovery period of rest following Challenge 3), an end-of-test BP measurement (hp_end) and an end-of-test heart rate measurement (hr_end) are taken and the results are provided to the storage device 120.

In one or more embodiments, throughout the OSA screen, or beginning after the baseline BP test and continuing to the end of the OSA screen, a pulse oximeter is worn on the subject's finger and measures the subject's heart rate and blood oxygen saturation at a sampling rate of, e.g., about 3 Hz.

In some embodiments, the disclosed system further performs an OSA screen prediction based on the collected information in the storage device 120. For example, feature extraction may be a first stage in an OSA screen prediction according to embodiments of the present disclosure. Feature extraction extracts a useful set of features from the collected information. An effective set of extracted features can decrease a computational complexity of the system and increase subsequent classification performance.

In some embodiments, five categories of features are used in the OSA screen prediction: physiological, sleep quality-related, sleepiness-related, psychological and contextual features.

Category I. In some embodiments, physiological measurements used in the analysis include bp_baseline, hr_baseline, bp_resting, hr_resting, bp_end, hr_end, and blood oxygen saturation (Sp0₂), among others. In some embodiments, features extracted from the physiological measurements are: a difference between systolic BP measurements taken during the rest periods M1 and M2 (bp_sys_di), a difference between diastolic BP measurements taken during the rest periods M1 and M2 (bp_dias_di), and a difference between the heart rate measurements taken during the rest periods M1 and M2 (HR_di). The Valsalva maneuver performed in phase IV of the protocol may evoke blood pressure and heart rate changes. For example, these changes can involve a rapid increase in blood pressure at the time of exhalation (BP rises above baseline), followed by a rapid decrease in blood pressure at the time of air pressure release when the user starts breathing normally (BP falls below baseline) and then, recovery (BP goes back to baseline). The heart rate may show a similar response but in the opposite direction of blood pressure.

Although heart rate and blood pressure changes occur in healthy subjects as well as OSA sufferers, a response of an OSA sufferer is delayed and less pronounced. In some embodiments, the three aforementioned extracted difference measurement features may capture the extent of change in blood pressure and heart rate between the baseline measurement taken during the rest period M1 and the post-Valsalva measurement taken during the rest period M2 to help distinguishing between OSA-suffering and healthy persons.

Category II. In some embodiments, the system extracts sleep quality-related features based on, e.g., sleep efficiency, sleep latency and sleep duration.

Sleep efficiency (sleep_efficiency) is a measure of sleep quality. In some embodiments, OSA sufferers may have a high sleep efficiency because, due to sleep deprivation, they fall asleep quickly and stay asleep for most of the time while they are in bed. However, the OSA sufferers' sleep may not be in REM (rapid eye movement) and NREM3 (non-REM stage 3) sleep, which are the sleep stages during which the physical and mental regeneration occurs, respectively.

In some other embodiments, some OSA sufferers, however, can have a low sleep efficiency if they suffer from depression, which may be a comorbidity of OSA. Insomnia, a symptom of depression, can cause OSA sufferers to have a sleep efficiency lower than that of a healthy person. The low sleep efficiency can negatively affect sleepiness (explained under Category III). In some embodiments, a scoring scheme used to differentiate among 4 different levels of sleep efficiency may be, e.g., >about 85% (very good), about 75%-about 85% (good), about 65%-about 74% (bad), and <about 65% (very bad). Sleep efficiency may be derived from answers to the interrogatories. In one or more embodiments, sleep_efficiency is defined as the quantity (total time spent sleeping)/(total time spent in bed). For example, sleep_efficiency may be calculated for the questions of Table 1 as Q3/(Q4-Q1), where Q3, Q4, Q1 are numbers given in response to the questions q_3, q_4 and q_1, respectively.

Sleep latency (sleep_latency) is a measure of sleep deprivation. Sleep latency is the amount of time it takes a person to fall asleep. Due to their disorder, OSA sufferers may be sleep deprived and fall asleep much faster than a healthy person. In other words, OSA sufferers may have a relatively shorter sleep latency than healthy persons. In some embodiments, a scoring scheme used to differentiate among 4 different levels of sleep latency may be, e.g., <=about 15 minutes (sleep deprived), about 16-about 30 minutes (normal), about 31-about 60 minutes (long normal), and >about 60 minutes (insomniac). Sleep latency may be derived from answers to the interrogatories. For example, sleep_latency may be calculated for the questions of Table 1 as Q2, which is a number given in response to question q_2.

Sleep duration (sleep_duration) may be derived from answers to the interrogatories. For example, sleep_duration may be calculated for the questions of Table I as Q3, which is a number given in response to question q_3. A scoring scheme used to differentiate among 4 different levels of sleep duration may be, e.g., >about 7 hours, about 6-about 7 hours, about 5-about 6 hours, and <about 5 hours. OSA sufferers may have relatively longer sleep durations relative to healthy persons because poor sleep quality does not provide the rest that OSA sufferers need, causing them to stay in bed longer.

Category III. In some embodiments, extracted features include a measure of daytime sleepiness, which may be calculated for the questions of Table I as Q5, which is a number given in response to question q_5. OSA sufferers may have higher sleepiness values relative to healthy persons because OSA results in excessive daytime sleepiness.

Category IV. In some embodiments, the features used in the OSA screen prediction include psychological features include perceived stress, depression, anxiety and psychological distress. Due to the negative effects of sleep deprivation on the human psychology, OSA sufferers may have worse patient psychology than non-OSA persons.

Perceived stress is a measure of a degree to which a person is experiencing stress in life. Perceived stress may be derived from answers to the interrogatories. For example, perceived stress may be calculated for the questions of Table I as a function of Q7, Q8, Q9 and Q10, which are respectively numbers given in response to questions q_7, q_8, q_9 and q_10 (e.g., perceived stress may be calculated as Q7+Q10+4−(Q8+Q9)). In an embodiment, each of the perceived stress questions may have five response alternatives: 0=never, 1=almost never, 2=sometimes, 3=fairly often, and 4=very often.

Depression is derived from answers to interrogatories. For example, depression may be calculated for the questions of Table I as Q11+Q12, where Q11, Q12 are respectively numbers given in response to questions q_11, q_12. In an embodiment, each of the depression questions may have four response alternatives: 0=not at all, 1=several times, 2=very often, and 3=the whole day, and scoring can be depressive (3-4) and normal (0-2).

Anxiety may be derived from answers to the interrogatories. For example, anxiety may be calculated for the questions of Table I as Q13+Q14, where Q13, Q14 are respectively numbers given in response to questions q_13, q_14. In an embodiment, each of the anxiety questions may be four response alternatives: 0=not at all, 1=several times, 2=very often, and 3=the whole day, and scoring can be anxious (3-4) and normal (0-2).

In some embodiments, psychological distress may be a global measure based on a combination of the depression and anxiety scores, and may be calculated by adding the depression and anxiety scores (depression+anxiety). In an embodiment, scoring can be normal (0-2), mild (3-5), moderate (6-8), and severe (9-12).

Category V. In some embodiments, the features used in the OSA screen prediction include contextual features including atypical events and physical activity.

In some embodiments, atypical events may be a measure of whether or not the subject has experienced anything out of their normal routine in the previous 24 hours, whether good news, bad news, or an atypical event. The metric of atypical events may be derived from answers to the interrogatories. For example, atypical events may be calculated for the questions of Table I as Q6, which is a number given in response to question q_6. This question may help to understand the subject's daily context and may be useful for eliminating outliers, because atypical events may elicit OSA symptoms in non-OSA persons. A non-OSA subject who just got fired, for example, may exhibit the same symptoms of depression, anxiety and poor sleep quality as an OSA sufferer.

Physical activity may be a measure of a subject's energy level. An increased physical activity can result in improved happiness and decreased stress and anxiety. The metric of physical activity may be derived from answers to the interrogatories. For example, physical activity may be calculated for the questions of Table I as Q15, which is a number given in response to question q_15.

Following feature extraction, classification of the extracted features is performed. The extracted features are provided to a classifier as input. The classifier outputs a decision as to whether or not a person is suffering from OSA.

In an embodiment of the classification technique, a feature vector including the abovementioned features for a specific subject on a specific day is provided to the classifier as input. The classifier maps the input to one of the two possible output classes of OSA or non-OSA. A training dataset is used to train the classifier and to obtain parameters that will provide the best classification performance. The training dataset may contain multiple instances of feature vectors and their corresponding class labels. A separate testing dataset contains unlabeled feature vectors to which the classifier is to assign class labels. The overall accuracy of the classifier is determined by the percentage of the data in the testing dataset that is assigned to the correct output class. In some embodiments, the classifier for the OSA prediction technique may be, e.g., a support vector machine (SVM) classifier or a k-nearest-neighbor (k-NN) classifier.

An SVM classifier is a binary non-probabilistic linear classifier. Training an SVM includes constructing an optimal hyperplane in the feature space. The optimal hyperplane is one that maximizes a separation between the nearest training samples of the two different classes. Once the hyperplane is constructed, the testing data can be projected onto the feature space and features can be classified by the hyperplane into one of the two classes. In an experiment (experimental results below), a sigmoid kernel function may be used with a cost of about 0.8 and an intercept constant of about 0.1.

A k-NN classifier uses the notion of distance between data points in a feature space as the basis for classification. In a training phase, the labeled feature vectors are stored with the class labels of the training samples. In a testing phase, the k-NN classifier assigns a testing data point to the class that is the most common among the testing point's k nearest neighbors based on a majority vote of its neighbors. In an experiment (experimental results below), k may be set to three (k=3) and the Manhattan distance may be used as a distance metric.

In an embodiment, the triggering technique is implemented in part using an application (“OSA App”) supported by an operating system (e.g., Android or iOS). However, it should be understood that the concepts described herein may be implemented within other operating systems. The OSA App may include a user interface for providing instructions and interrogatories, and for receiving responses.

### Experimental Results I

According to an embodiment of the present disclosure, an OSA screen system was deployed in a pilot study of sixteen subjects. Three of the subjects had been previously diagnosed with sleep apnea through PSG and the remaining thirteen were control subjects. All subjects were given kits including an Android phone preloaded with an OSA App, a Bluetooth-enabled BP monitor, a Bluetooth-enabled pulse oximeter, and a Valsalva device. Each subject completed the test protocol (e.g., as described above with respect to FIG. 2) daily for a total period of 42 days. The data recorded by the OSA App was uploaded to a HIPAA-compliant server at the completion of each daily test. The uploaded data was then split into a training set and a testing set. The training set was used by the prediction system to train the classifiers, while the testing set was used as input to the trained classifiers to make predictions and to evaluate the performance of the classifiers.

To evaluate the performance of the classifiers, testing was performed using a model validation process such as Leave-One-Out-Cross-Validation (LOOCV), which evaluated the accuracy of the classifiers without bias. In some embodiments, the data included 42 observations per subject, an n-fold cross-validation approach may yield overly-optimistic results since a subset of those observations would be in the training set and the rest of the observations would be in the testing set. Having observations for the same subject in both the training and testing sets may introduce bias into the results because it is expected that observations for the same subject will be similar even if the test was performed on different days.

In some embodiments, a special case of LOOCV was employed; for example, a leave-one-patient-out-cross-validation (LOPOCV) in which observations for one subject were omitted and the classifier was trained on the remaining data. The omitted subject's observations were then used for testing. After filtering out outliers, 424 observations for the non-OSA class and 102 observations for the OSA class remained. Due to the imbalance in the dataset (3 OSA sufferers with 102 observations versus 13 control subjects with 424 observations), the training dataset was randomly under-sampled to balance the OSA and non-OSA observations.

Using the extracted features described above and LOPOCV, an overall classification accuracy was achieved of 75.1% and 79.8% using the k=3 NN and SVM classifiers, respectively. Table 2 shows the confusion matrices for the two classifiers.

TABLE II

| | Predicted Outcome | | | |
|---|---|---|---|---|
| | SVM | | k = 3 NN | |
| OSA? | Yes | No | Yes | No |
| Yes | 68 | 34 | 80 | 22 |
| No | 72 | 352 | 109 | 315 |
| Sensitivity/Specificity | 66.7% | 83.0% | 78.4% | 74.3% |

| | | |
|---|---|---|
| Overall Accuracy | 79.8% | 75.1% |

Even though the overall accuracy of the SVM classifier is nearly 80% as compared to the nearly 75% accuracy of the k=3 NN classifier, the sensitivity of SVM is lower than the sensitivity of k=3 NN. In some embodiments, for a medical classification task, it is desired for the results to be both sensitive (all OSA sufferers are correctly classified as OSA) and specific (all healthy subjects are correctly classified as healthy) because it is undesirable for people with OSA to go unnoticed or people without OSA to be improperly diagnosed. With 78.4% sensitivity and 74.3% specificity and an overall screening accuracy of 75.1%, the k=3 NN classifier is suitable for this classification task.

The SVM classifier builds a global model of the data, meaning that it uses the complete training dataset to compute a global function that maps a feature vector to a class label. The k-NN classifier, on the other hand, takes a local approach to classification where prediction is done by local functions using a subset of the training dataset, e.g., the neighboring points in this case. In some embodiments, the local approach may perform better than the global one for the prediction of OSA. The reason may be that not all members of the same class, whether OSA or non-OSA, are similar to each other, but rather that within the same class there are different clusters of subjects. For example, some healthy subjects can have poor sleep quality and some OSA sufferers can be psychologically healthier than non-OSA subjects, but in both cases, the healthy subjects and the OSA sufferers may share other properties with subjects from their class. The SVM constructs a hyperplane that partitions the feature space into two regions and thus, The SVM may tend to oversimplify the two classifications. The local approach works well because decisions are made based on small local neighborhoods of similar clusters, which assigns more significance to differences between subjects in the same class.

### Experimental Results II

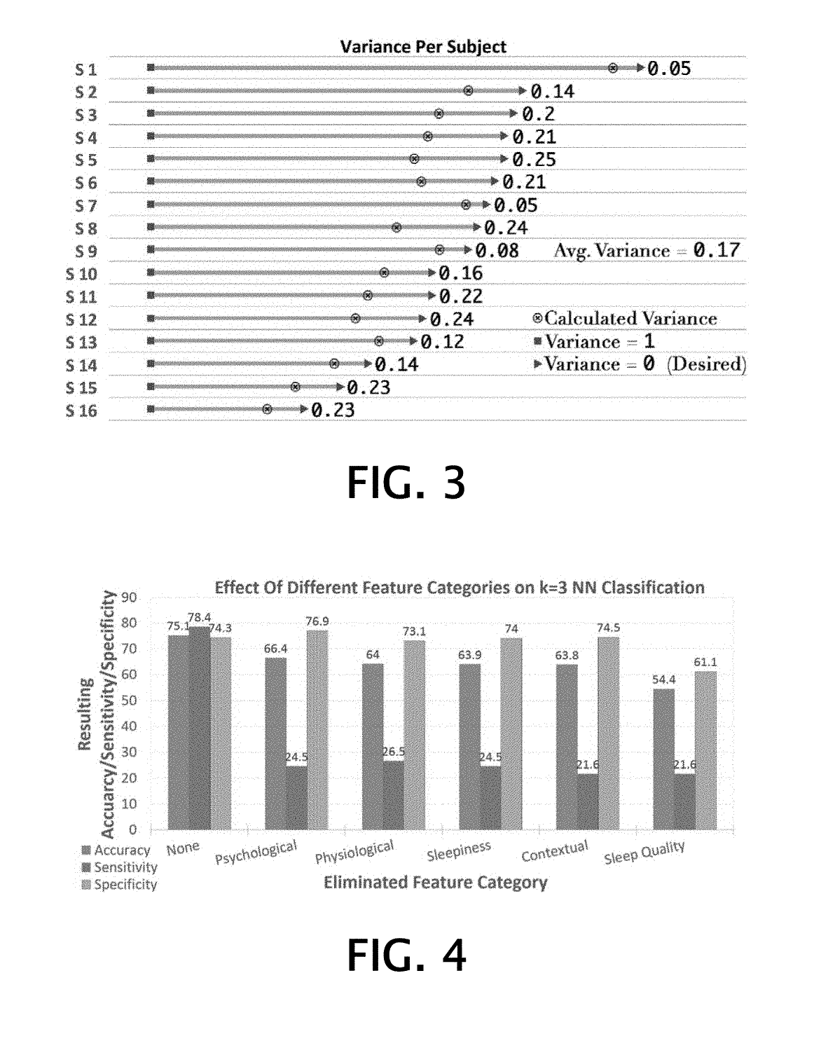

Another metric for evaluating a classifier, especially one that is applied in the medical field, is variance. In the Experimental Results I section above, a test was described in which each subject repeated the screening test daily for a period of 42 days. Though the answers to the questionnaire and the BP and heart rate measurements may vary from day to day for the same subject, it is not expected that the intra-subject variability will be high, or the classification as to whether or not a subject is suffering from OSA to change from one day to the next. Therefore, the ideal classifier would assign all the observation for the same subject to the same class label. The repeatability of a medical test is desirable for its validity and reliability. To measure the repeatability of the test, statistical variance may be used to measure variation in the predictions of the classifiers; the lower the variance, the more consistent the predictions are and the more repeatable the test is. FIG. 3 shows the variance for each subject's predictions and the overall average variance of the k=3 k-NN classifier. Notice that a test may have a perfect (zero) variance if it always gives the wrong prediction (or always gives the right prediction). Here, the classification results are both accurate (75.1% accuracy) and repeatable (0.17 variance).

### Experimental Results III

In an attempt to better understand the importance of each feature category for the prediction of OSA, one feature category was successively eliminated from the dataset in turn, and the training and testing repeated for each reduced dataset using the k=3 k-NN classifier. FIG. 4 shows the accuracy, sensitivity, and specificity of the classification after eliminating each of the five feature categories. The experimental results, as shown in FIG. 4, indicate a decrease in classification accuracy when any of the five feature categories is removed, compared to when all categories are preserved. The decrease in accuracy is mainly due to the dramatic decrease in sensitivity. As FIG. 4 shows, when any one of the five feature categories is missing, the test's ability to correctly identify OSA sufferers diminishes from 78%, when all features are present, to 24% on average when one of the categories is missing. This shows that, at least in the embodiment as illustrated, the feature categories play a role in the diagnosis of OSA. The results reveal the intricacy of the OSA disorder which affects first sleep quality, and as a result, body, mind and daily activities.

### Experimental Results IV

Though OSA is more prevalent in males, studies have shown that it can also have detrimental effect on females. In some embodiments, females show a more pronounced heart rate response impairment (lower amplitude, delayed onset, and slower rate changes) than males. Given the different effects that OSA has on members of the different genders and the different resulting heart rate response patterns, in some embodiments, two different prediction models may be used for male and female subjects respectively. Features can also be selected for each gender group separately. To make a diagnosis, a subject may be first clustered into his/her appropriate gender group and then classification may be performed using the specific model for the subject's gender. This gender-aware classification may achieve superior results to the gender-agnostic model. In some embodiments, a Bayesian Network classifier achieves good results for the male cluster, while the k-NN classifier achieves good results for the female cluster.

A Bayesian Network is a probabilistic graphical model in which the feature space is represented as a directed acyclic graph (DAG). The nodes of the DAG correspond to the features, while the edges of the DAG represent the influence of one feature on another. Each node is annotated with a conditional probability distribution that represents p(XijPa(Xi)), where Xi is a feature and Pa(Xi) is the parent of that feature in the DAG. Probabilistic inference can then be used to make predictions about the class to which a feature vector belongs.

The k-NN classifier may defer all computation until after classification. In the training phase, a k-NN classifier stores the feature vectors and class labels, and in the testing phase, assigns a testing data point to the class label of the majority of its k nearest neighbors based on a distance metric. In the experiment as illustrated, k=3 and the distance metric is the Manhattan distance.

To avoid pseudo-replication, LOPOCV may be used, where all observations for one subject were used for testing and the remaining observations are used to train the gender-specific models.

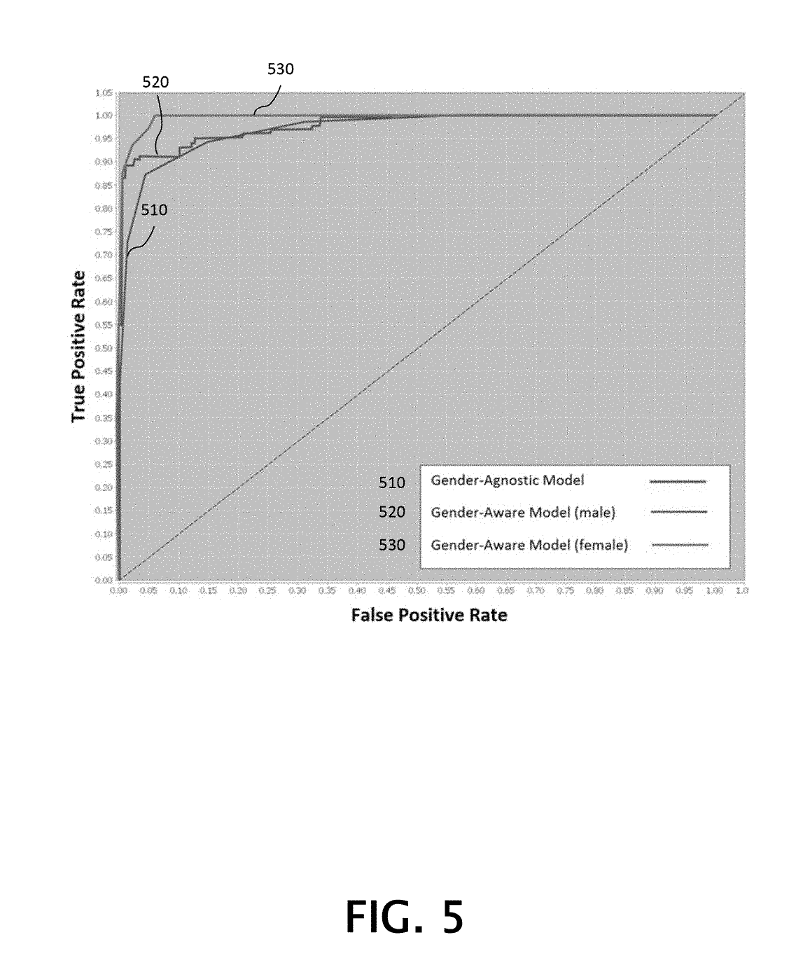

FIG. 5 shows Receiver Operating Characteristics (ROC) curves for gender-agnostic and gender-aware OSA classifications, to evaluate the merit of a gender-aware model for the diagnosis of OSA. In some embodiments, the classifier that achieves good results for the entire dataset (male+female subjects) may be a Random Forest classifier. The Random Forest classifier is an ensemble learning method that constructs multiple decision tree predictors at training time. Each tree casts a vote about the class label for a specific feature vector and the most popular class is assigned to the feature vector at testing time. As can be seen in FIG. 5, both the gender-agnostic (510) and gender-aware models (520, 530) achieve excellent prediction accuracy (AUC>0.97) as a result of the disclosed screening protocol and the extracted physiological features. The benefit of a gender-aware approach shows when comparing the sensitivity of the classification. Table III shows a more detailed evaluation of the gender-aware and gender agnostic approaches. While the gender-aware models are successful at detecting OSA sufferers over 90% of the time, the gender-agnostic model is successful 74% of the time. Thus, in some embodiments, the gender-aware approach may be more suitable for a medical diagnostic test. In some embodiments, on a closer investigation, for more than 90% of the OSA instances in which the gender-agnostic approach misclassified a subject as Control, the subject was male. A possible explanation is that, when one model is used for males and females, the male OSA sufferers appear healthy compared to their female counterparts who, as stated earlier, are more seriously affected by OSA.

TABLE III

| | | | Area Under | Overall |
|---|---|---|---|---|
| Model | Sensitivity | Specificity | ROC | Accuracy |
| Gender-Agnostic | 0.736 | 0.989 | 0.972 | 91.8% |
| Gender-Aware (M) | 0.915 | 0.968 | 0.977 | 94.8% |
| Gender-Aware (F) | 0.933 | 0.984 | 0.995 | 97.7% |

In some embodiments, in addition to classifying subjects into Control and OSA classes, a more fine-grained analysis may be used to classify subjects by severity levels of the OSA disorder (mild, moderate, and severe).

Therefore, herein describe an affordable, time efficient, and non-nocturnal OSA screening system and technology. Accuracy of the proposed OSA screen classification system is validated and the results demonstrate ability in assisting medical personnel in assessment of subjects with suspected OSA.

In some embodiments, the OSA screen system of the present disclosure may be implemented as computer-executable instructions in a memory, executed by a computing device. Accordingly, a computing device may be configured to execute instructions from a memory, such as to provide interrogatories at a user interface; receive responses to the interrogatories at the user interface; receive blood pressure measurement information from the blood pressure monitoring device; receive heart rate measurement information from the heart rate monitoring device; and determine, from the responses to the interrogatories, the blood pressure measurement information and the heart rate measurement information, a classification of a subject, the classification being either having obstructive sleep apnea disorder or not having obstructive sleep apnea disorder.

Although various embodiments disclose obstructive sleep apnea (OSA) as examples, it is to be understood that the disclosed screening system and technology may be applied to any types of sleep apnea such as obstructive sleep apnea, central sleep apnea, or mixed sleep apnea.

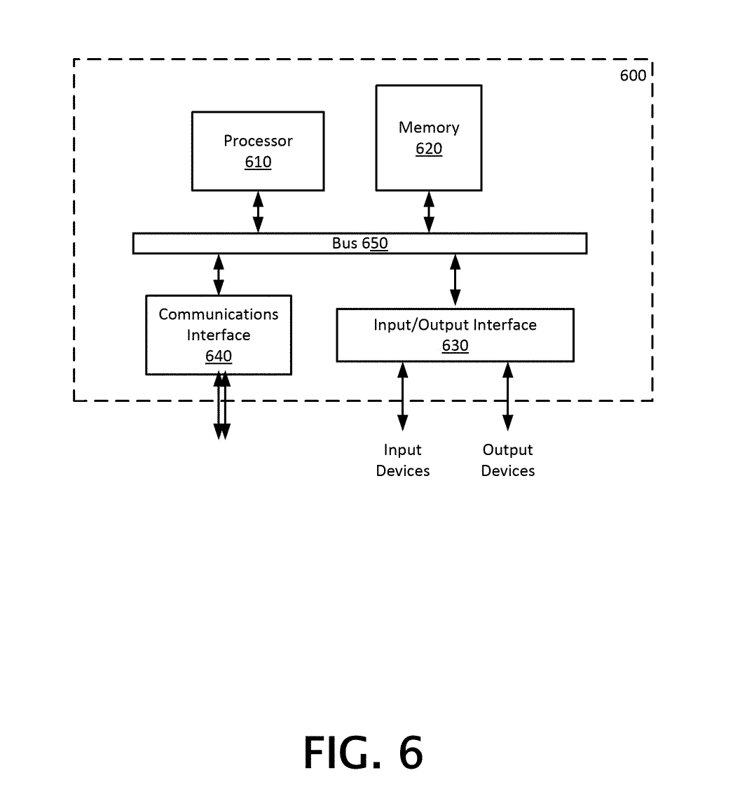

FIG. 6 illustrates an example of a computing device 600 (e.g., computing device 110) that includes a processor 610, a memory 620, an input/output interface 630, and a communication interface 640. A bus 650 provides a communication path between two or more of the components of computing device 600. The components shown are provided by way of illustration and are not limiting. Computing device 600 may have additional or fewer components, or multiple of the same component.

Processor 610 represents one or more of a processor, microprocessor, microcontroller, ASIC, and/or FPGA, along with associated logic.

Memory 620 represents one or both of volatile and non-volatile memory for storing information. Examples of memory include semiconductor memory devices such as EPROM, EEPROM, RAM, and flash memory devices, discs such as internal hard drives, removable hard drives, magneto-optical, CD, DVD, and Blu-ray discs, memory sticks, and the like.

Portions of the OSA screen system of this disclosure may be implemented as computer-readable instructions in memory 620 of computing device 600, executed by processor 610.

Input/output interface 630 represents electrical components and optional code that together provides an interface from the internal components of computing device 600 to external components. Examples include a driver integrated circuit with associated programming.

Communications interface 640 represents electrical components and optional code that together provides an interface from the internal components of computing device 600 to external networks, such as network 115.

Bus 650 represents one or more interfaces between components within computing device 600. For example, bus 650 may include a dedicated connection between processor 610 and memory 620 as well as a shared connection between processor 610 and multiple other components of computing device 600.

An embodiment of the disclosure relates to a non-transitory computer-readable storage medium having computer code thereon for performing various computer-implemented operations. The term “computer-readable storage medium” is used herein to include any medium that is capable of storing or encoding a sequence of instructions or computer codes for performing the operations, methodologies, and techniques described herein. The media and computer code may be those specially designed and constructed for the purposes of the embodiments of the disclosure, or they may be of the kind well known and available to those having skill in the computer software arts. Examples of computer-readable storage media include, but are not limited to: magnetic media such as hard disks, floppy disks, and magnetic tape; optical media such as CD-ROMs and holographic devices; magneto-optical media such as optical disks; and hardware devices that are specially configured to store and execute program code, such as application-specific integrated circuits (“ASICs”), programmable logic devices (“PLDs”), and ROM and RAM devices.

Examples of computer code include machine code, such as produced by a compiler, and files containing higher-level code that are executed by a computer using an interpreter or a compiler. For example, an embodiment of the disclosure may be implemented using Java, C++, or other object-oriented programming language and development tools. Additional examples of computer code include encrypted code and compressed code. Moreover, an embodiment of the disclosure may be downloaded as a computer program product, which may be transferred from a remote computer (e.g., a server computer) to a requesting computer (e.g., a client computer or a different server computer) via a transmission channel. Another embodiment of the disclosure may be implemented in hardwired circuitry in place of, or in combination with, machine-executable software instructions.

As used herein, the singular terms “a,” “an,” and “the” may include plural referents unless the context clearly dictates otherwise.

As used herein, the terms “approximately,” “substantially,” “substantial” and “about” are used to describe and account for small variations. When used in conjunction with an event or circumstance, the terms can refer to instances in which the event or circumstance occurs precisely as well as instances in which the event or circumstance occurs to a close approximation. For example, when used in conjunction with a numerical value, the terms can refer to a range of variation less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, two numerical values can be deemed to be “substantially” the same if a difference between the values is less than or equal to ±10% of an average of the values, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified.

While the present disclosure has been described and illustrated with reference to specific embodiments thereof, these descriptions and illustrations do not limit the present disclosure. It should be understood by those skilled in the art that various changes may be made and equivalents may be substituted without departing from the true spirit and scope of the present disclosure as defined by the appended claims. The illustrations may not be necessarily drawn to scale. There may be distinctions between the artistic renditions in the present disclosure and the actual apparatus due to manufacturing processes and tolerances. There may be other embodiments of the present disclosure which are not specifically illustrated. The specification and drawings are to be regarded as illustrative rather than restrictive. Modifications may be made to adapt a particular situation, material, composition of matter, method, or process to the objective, spirit and scope of the present disclosure. All such modifications are intended to be within the scope of the claims appended hereto. While the methods disclosed herein have been described with reference to particular operations performed in a particular order, it will be understood that these operations may be combined, sub-divided, or re-ordered to form an equivalent method without departing from the teachings of the present disclosure. Accordingly, unless specifically indicated herein, the order and grouping of the operations are not limitations of the present disclosure.

What is claimed is:

1. A method of sleep apnea diagnosis, comprising:
providing interrogatories at a user interface;
receiving responses to the interrogatories at the user interface;
receiving blood pressure measurement information from a blood pressure monitoring device;
receiving heart rate measurement information from a heart rate monitoring device; and
determining, from the responses to the interrogatories, the blood pressure measurement information and the heart rate measurement information, a classification of a subject, the classification being either having sleep apnea disorder or not having sleep apnea disorder.

2. The method of claim 1, further comprising:
prompting to conduct a challenge activity while continuing to receive the blood pressure measurement information and the heart rate measurement information.

3. The method of claim 2, wherein the challenge activity simulates an apneic attack and triggers changes in the heart rate measurement information and the blood pressure measurement information that occur after the apneic attack.

4. The method of claim 2, wherein the challenge activity is a psychological and cognitive stressor inducing heightened levels of physiological responses.

5. The method of claim 2, wherein the challenge activity includes a Stroop test, a Valsalva maneuver, or a breath holding exercise.

6. The method of claim 1, wherein the determining the classification comprises:
extracting features from the responses to the interrogatories, the blood pressure measurement information and the heart rate measurement information; and
wherein the extracted features comprise a change between a blood pressure measurement or a heart rate measurement at a first period prior to a challenge activity and a blood pressure measurement or a heart rate measurement at a second period subsequent to the challenge activity.

7. The method of claim 1, wherein the determining the classification comprises:
determining that the change between the blood pressure measurement or the heart rate measurement at the first period and the blood pressure measurement or the heart rate measurement at the second period is delayed or less pronounced, compared to a change for a healthy person.

8. The method of claim 1, wherein the determining the classification comprises:
extracting features from the responses to the interrogatories, the blood pressure measurement information and the heart rate measurement information; and
wherein the extracted features comprise scores for sleep quality-related, sleepiness-related, psychological and contextual features.

9. The method of claim 1, further comprising:
receiving blood oxygen information from an oximeter device.

10. The method of claim 1, wherein the classification of the subject is determined by a support vector machine classifier or a k-nearest-neighbor classifier.

11. The method of claim 1, wherein a first interrogatory of the interrogatories requests an identification of gender as female or male, and the determining the classification comprises selecting a first classifier in response to an identification of the gender being female and to select a second classifier in response to an identification of the gender being male.

12. The method of claim 11, wherein the first classifier is a k-nearest-neighbor classifier and the second classifier is a Bayesian network classifier.

13. The method of claim 1, wherein the sleep apnea disorder is an obstructive sleep apnea, a central sleep apnea, or a mixed sleep apnea.

14. A system for sleep apnea diagnosis, comprising:
a user interface configured to provide interrogatories and receive responses to the interrogatories;
a blood pressure monitoring device configured to collect blood pressure measurement information;
a heart rate monitoring device configured to collect heart rate measurement information;
a data storage component configured to store the responses to the interrogatories, the blood pressure measurement information, and the heart rate measurement information;
a classifier configured to determine a classification of a subject, the classification being either having sleep apnea disorder or not having sleep apnea disorder, based on the responses to the interrogatories, the blood pressure measurement information and the heart rate measurement information.

15. The system of claim 14, further comprising:
an oximeter device configured to receive blood oxygen information.

16. The system of claim 14, wherein the user interface is further configured to prompt a user to conduct a challenge activity that triggers changes in the heart rate measurement information and the blood pressure measurement information.

17. The system of claim 14, further comprising:
a feature extractor configured to extract features from the responses to the interrogatories, the blood pressure measurement information and the heart rate measurement information;
wherein the extracted features comprise scores for sleep quality-related, sleepiness-related, psychological and contextual features.

18. The system of claim 14, wherein the classifier is trained using a training dataset, the training dataset includes instances of feature vectors and classifications corresponding to the instances of feature vectors.

19. The system of claim 14, wherein a first interrogatory of the interrogatories requests an identification of gender as female or male, and a first instance of the classifier is in response to an identification of the gender being female and a second instance of the classifier is in response to an identification of the gender being male.

20. A sleep apnea diagnostic system, comprising:
a computing device configured to execute instructions from a memory to:
provide interrogatories at a user interface;
receive responses to the interrogatories at the user interface;
receive blood pressure measurement information from a blood pressure monitoring device;
receive heart rate measurement information from a heart rate monitoring device; and
determine, from the responses to the interrogatories, the blood pressure measurement information and the heart rate measurement information, a classification of a subject, the classification being either having sleep apnea disorder or not having sleep apnea disorder.
